(19) United States
(12) Patent Application Publication
(10) Pub. No.: US 2005/0069899 A1
(43) Pub. Date: Mar. 31, 2005

(54) Method of synthesizing a target polynucleotide efficiently expressed in a host-vector expression system

(75) Inventors: Chao-Wei Liao, Chunan Town (TW); Shin-Hung Lin, Chunan Town (TW); Chi-Min Chen, Chunan Town (TW); Chung-Nan Weng, Chunan Town (TW)

Correspondence Address: Ladas & Parry, 26 West 61th Street, New York, NY, 10023, US

(73) Assignee: ANIMAL TECHNOLOGY INSTITUTE TAIWAN

(21) Appl. No.: 10/672,746

(22) Filed: Sep. 26, 2003

(51) Int. Cl. C12Q 1/68; C12P 19/34

(52) U.S. Cl. 435/6; 435/91.2

(57) ABSTRACT

The present invention provides a method of synthesizing a target polynucleotide that is efficiently expressed in a host-vector expression system, which uses a primer extension technique to constitute the target polynucleotide sequence. Preferably, the method is applied in a method for highly expressing a target heterogeneous polypeptide encoded by the target polynucleotide in a host.

### BACKGROUND OF THE INVENTION

1. Field of the Invention

The invention mainly relates to a method of synthesizing a target polynucleotide efficiently expressed in a host-vector expression system.

2. Description of the Related Art

Synthesis of a polynucleotide having a particular or given polynucleotide sequence is important to life science exploration. Such particular polynucleotide sequence usually encodes a protein, and especially, encodes a heterogeneous protein for expressing in a host cell. In conventional methods of synthesis of a known oligonucleotide having a particular sequence shorter than about 50 nucleotides, a chemical synthesis is used. However, it is difficult to synthesize a larger polynucleotide fragment through a chemical synthesis and the manipulation is complicated.

Generally, polymerase chain reaction (PCR) is a usual method for amplifying a large polynucleotide fragment in vitro (Kleppe K. Ohtsuka E., Kleppe R., Molineux I. and Khorana H. G. 1971. Studies on polynucleotide. XCVI. Repair replication of short synthetic DNA is catalyzed by DNA polymerases. J. Mol. Biol. 56: 341-361). PCR usually comprises four steps: (1) denaturaling a template to form two single strands; (2) annealing two primers to the two strands in the step (1), respectively; (3) extending the primers by DNA polymerase; and (4) obtaining two double strands of DNAs. The steps mentioned above are repeated, and a particular DNA fragment is amplified. To conduct a PCR, the following materials are needed: (a) a template which comprises a DNA fragment of the particular DNA sequence to be produced; (b) a pair of primers which hybrid the two strands of the template at the 5′-ends of the two strands, respectively; (c) DNA polymerase(s) and dNTP for synthesizing under proper conditions.

Some methods of PCR are suitable for generating a polynucleotide having a little modifications relative to a template thereof such as a site-directed mutagenesis through a PCR (Ho S. N., Hunt H. D., Horton R. M., Pullen J. K., and Pease L. R. 1989. Site-directed mutagenesis by overlap extension using the polymerase chain reaction. Gene 77: 51-59). In the site-directed mutagenesis through a PCR, one or more nucleotides can be designed in a primer for being added, substituted or deleted in a wild type template sequence. However, if there are many mutation sites generated in one primer, the PCR cannot be successfully performed. The reason would be that a primer with many mutation sites leads a non-specific hybridization with the template. In a conventional PCR for generating many mutation sites in a small region, such as within 60 base pairs, a first stage of the PCR is conducted to generate some mutation sites with the use of the wild type sequence as a template. A product of the first stage of the PCR should be purified and then used as a template in a second stage of the PCR to generate some mutation sites on the product of the first stage of the PCR. A product of the second stage of the PCR should be purified and then used as a template in a third stage of the PCR to generate other mutation sites on the product of the second stage of the PCR. In most stages of the PCR, it may be necessary to generate mutation sites to the wild type sequence. Therefore, the manipulation is complicated and laborious.

Besides, a template molecule that is highly homologous to the product is required. However, if no original template can be obtained in some situations, there is no way to obtain the product by a conventional PCR.

In another aspect, some heterogeneous proteins cannot be expressed in a host-vector system because of the difference in codon usage between the protein original species and the host species. For example, a codon for tryptophan in human mitochondria or Mycoplasma spp. is UGA, but it is a stop signal in an Escherichia coli host-vector system. Therefore, in most cases, the protein expression yield is very low or dissatisfied. Changing the codons seems to be a possible solution to the problem. For that matter, such codon change needs to generate multiple point mutation sites interspersed in a polynucleotide coding the heterogeneous protein comparing with the original gene obtained. However, for the above-mentioned reasons, it is difficult to obtain a desired result.

Therefore, an efficient and accurate method of synthesizing a target polynucleotide efficiently expressed in a host-vector expression system is still required.

### SUMMARY OF THE INVENTION

The present invention provides a method of synthesizing a target polynucleotide efficiently expressed in a host-vector expression system through a polymerase chain reaction (PCR). Preferably, the method is applied in a method for highly expressing a target heterogeneous polypeptide encoded by the target polynucleotide in a host. If the target polynucleotide sequence is heterogeneous to the host used in expressing the protein encoding the target polynucleotide, some codons of the target polynucleotide are changed to the codons which have a high expression efficiency in translating the same amino acid in the host cell.

One subject of the invention is to provide a method for synthesizing a target polynucleotide that is efficiently expressed in a host-vector expression system, comprising the steps of:
(1) conducting a first polymerase chain reaction on a first template with a first primer pair to obtain a first polymerase chain reaction product; which is characterized in that the first template is any template sequence commonly used in the host-vector expression system or a fragment of the target polynucleotide;
(2) conducting muti-cyclic polymerase chain reactions by a primer extension technique to obtain a product comprising the target polynucleotide sequence; wherein the template used in each polymerase chain reaction is the product obtained in the previous polymerase chain reaction; and
which is characterized in that the primer pairs used in the polymerase chain reactions are designed to be any one of the following three primer pairs:
(i) the forward primer having two parts:
   (a) the part (a1), locating at the 5′-end region of the forward primer, comprising a fragment having more than 10 nucleotides and being homologous to the fragment at the 3′-end region of the target polynucleotide sequence, and
   (b) the part (b1), locating at the 3′-end region of the forward primer, comprising a fragment having more than 10 nucleotides and being homologous to the sequence of the more than 10 nucleotides from the 5′-end region of the template sequence;
   and wherein the 3′-end of the part (a1) is adjacent to the 5′-end of the part (b1); and
the reversed primer having, at the 3′-end region of the reversed primer, a fragment having more than 5 nucleotides and being capable of annealing to the 3′-end region of the template sequence;
(ii) the forward primer having at the 3′-end region of the forward primer, a fragment having more than 5 nucleotides and being homologous to the 5′-end region of the template sequence; and
the reversed primer having
   (a) the part (a2), locating at the 5′-end region of the reversed primer, comprising a fragment having more than 10 nucleotides and being complement to the 5′-end region sequence of the target polynucleotide sequence;
   (b) the part (b2), locating at the 3′-end region of the reversed primer, comprising a fragment having more than 10 nucleotides and be capable of annealing to the sequence of the more than 10 nucleotides from the 3′-end region of the template sequence,
   and wherein the 3′-end of the part (a2) is adjacent to the 5′-end of the part (b2); and
(iii) the forward primer having
   (a) the part (a3), locating at the 5′-end region of the forward primer, comprising a fragment having more than 10 nucleotides and being homologous to the fragment at the 3′-end region of the target polynucleotide sequence;
   (b) the part (b3), locating at the 3′-end region of the forward primer, comprising a fragment having more than 10 nucleotides and being homologous to the sequence of the more than 10 nucleotides from the 5′-end region of the template sequence
   and wherein the 3′-end of the part (a3) is adjacent to the 5′-end of the part (b3); and
the reversed primer having
   (c) the part (c3), locating at the 5′-end region of the reversed primer, comprising a fragment having more than 10 nucleotides and being complement to the 5′-end region of the target polynucleotide sequence;
   (d) the part (d3), locating at the 3′-end region of the reversed primer, comprising a fragment having more than 10 nucleotides and annealing to the sequence of the more than 10 nucleotides from the 3′-end region of the template sequence;
   and wherein the 3′-end of the part (c3) is adjacent to the 5′-end of the part (d3); and
wherein all of the fragments of the target polynucleotide sequence used in the polymerase chain reactions in sequence constitute the target polynucleotide sequence; and
(3) obtaining the polynucleotide product comprising the target polynucleotide sequence from the final product of the muti-cyclic polymerase chain reactions.

The method according to the invention, if the target polynucleotide sequence is heterogeneous to the host used in expressing the protein encoding the target polynucleotide, some codons of the target polynucleotide are changed to the codons which have a high expression efficiency in translating the same amino acid in the host cell. In another aspect, the invention provides a method for highly expressing a target heterogeneous polypeptide encoded by a target polynucleotide in a host, which comprises the steps of:
(1) providing a target polynucleotide obtained by the method for synthesizing a target polynucleotide that is efficiently expressed in a host-vector expression system as described above;
(2) transforming or transfecting the target polynucleotide to the host; and
(3) expressing the target heterogeneous protein in the transformed and transfected host.

### BRIEF DESCRIPTION OF THE DRAWINGS

FIG. 1 illustrates the schematic figure of the method of synthesizing a target polynucleotide according to one embodiment of the invention.

FIG. 2 illustrates the schematic figure of the method of synthesizing a target polynucleotide according to another embodiment of the invention.

FIG. 3 illustrates the result of polymerase chain reaction product subjected to agarose gel electrophoresis when synthesizing the first template according to Example 1 of the invention.

FIG. 4 illustrates the result of polymerase chain reaction product subjected to agarose gel electrophoresis when synthesizing the polynucleotide product comprising the target polynucleotide sequence according to Example 1 of the invention.

FIG. 5 illustrates the schematic figure of Example 1 of the invention.

FIG. 6 illustrates the construction of PRRSV-ORF7 protein expression vector according to Example 1 of the invention.

FIG. 7 illustrates the expression result of FMD-vpg protein after changing codons subjected to SDS-PAGE according to Example 3 of the invention.

### DETAILED DESCRIPTION OF THE INVENTION

The present invention provides a method of synthesizing a target polynucleotide efficiently expressed in a host-vector expression system through a polymerase chain reaction (PCR). The method is initiated with a first template that is any template sequence commonly used in the host-vector expression system or a fragment of the target polynucleotide. The present invention is characterized in that a template that is highly relevant to the target polynucleotide is not necessary. Therefore, the polymerase chain reaction according to the invention can be used to produce various products, even without a template that is highly relevant to the target polynucleotide. Besides, according to the present invention, no purification step is required, and a PCR product obtained in the previous reaction can be directly used in a next reaction.

As used herein, the term “target polynucleotide sequence” refers to a sequence to be produced. The polynucleotide molecule corresponding to the target polynucleotide sequence may not be available or even may not exist in the nature. On the other hand, the target polynucleotide sequence may be a sequence coding for a protein or a peptide.

As used herein, the term “host-vector expression system” refers to a system comprising a host organism carrying a vector that contains a coding sequence for expressing a desired protein. Any conventional host organism convenient for maintenance and operation is suitable for practicing the invention. Preferably, the host organism is a microorganism. More preferably, the host organism is an enteric bacterium. The vector has an ability to express the protein in the host. According to the invention, a product can be expressed by a host-vector expression system where the vector is transformed with the target polynucleotide obtained according to the invention.

If the target polynucleotide sequence is heterogeneous to the host used in expressing the protein encoding the target polynucleotide, some codons of the target polynucleotide may be changed to the codons which have a high expression efficiency in translating the same amino acid in the host cell. The target polynucleotide sequence encoding a target protein may have multiple mutation sites comparing to the wild-type form thereof.

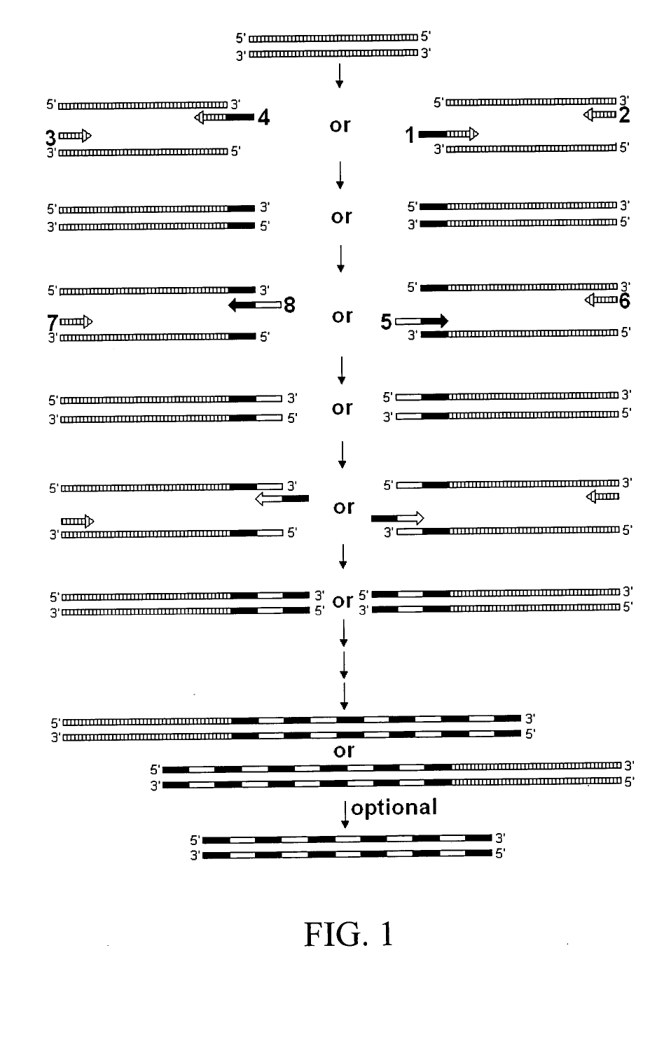

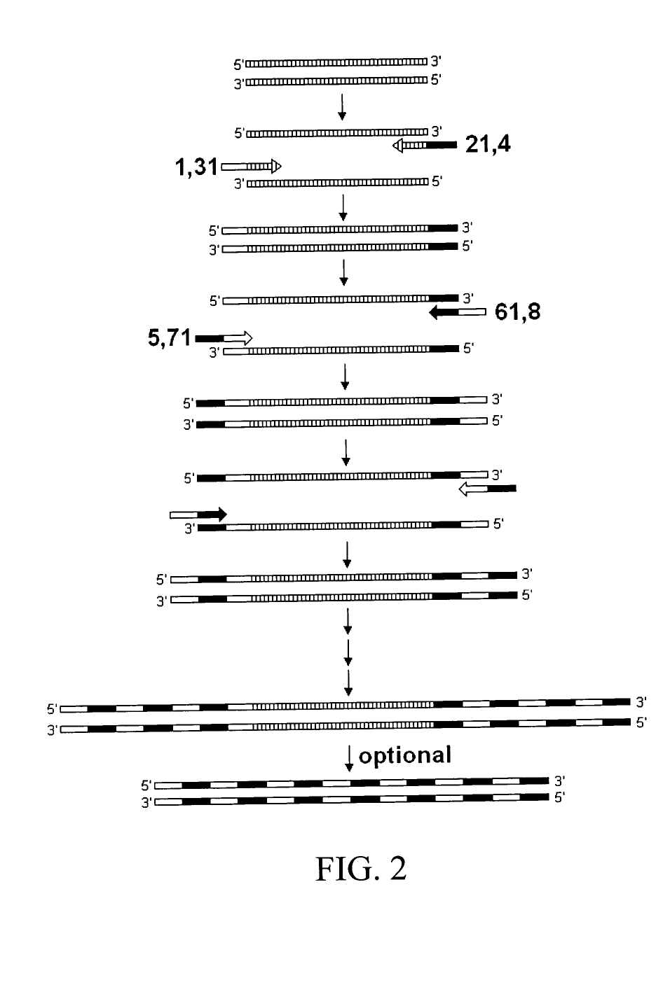

According to the invention, the method comprises the step (1) of conducting a first polymerase chain reaction on a first template with a first primer pair (as shown in FIGS. 1 and 2, primers 1, 2, 3, 4, 21, or 31) to obtain a first polymerase chain reaction (PCR) product; which is characterized in that the first template is any template sequence commonly used in the host-vector expression system or a fragment of the target polynucleotide.

As used herein, the term “template” refers to an oligonucleotide fragment used in a polymerase chain reaction for amplifying a molecule, which is the same or highly homologous to the molecule under the conditions ranging from moderate (about 5×SSC at 52° C.) to high (about 0.1×SSC at 65° C.) stringency conditions.

According to the invention, the template sequence commonly used in the host-vector expression system comprises, but is not limited to, a part or whole of a conventional vector, a gene fragment, a promoter fragment, or a polynucleotide fragment containing restriction enzyme recognition sites. In one embodiment of the invention, the first template is a conventional template used in synthesizing a polynucleotide sequence which is not relevant to the target polynucleotide sequence, when the polynucleotide molecule corresponding to the target polynucleotide sequence to be produced does not exist in the nature, or is not available.

In another aspect, the fragment of the target polynucleotide as the template according to the invention may be a polynucleotide sequence that encodes the protein heterogeneous to the host, where some codons thereof are changed to the codons which have a high expression efficiency in translating the same amino acid in the host cell, or which has multiple mutation sites comparing to the wild-type form thereof.

As used herein, the term “first primer pair” refers to an oligonucleotide fragment used for annealing to the first template in the first polymerase chain reaction. In one embodiment of the invention, a helper primer is provided, which is homologous to one primer of the first primer pair and identical to a fragment of one strand of the target polynucleotide. The homology between the helper primer and the template may not be enough to lead a successful annealing step. The first primer pair can anneal to the first template for carrying on some cycles in a polymerase chain reaction, and then the helper primer can anneal to a product made by the first primer pair for more cycles of the reaction. Preferably, the amount of the helper primer is more than that of the first primer pair.

According to the invention, the first polymerase chain reaction product provides a starting material for synthesizing the target polynucleotide, and wherein preferably, it also participates in constituting the target polynucleotide.

According to the invention, the method comprises the step (2) of conducting muti-cyclic polymerase chain reactions by a primer extension technique to obtain a product comprising the target polynucleotide sequence; wherein the template used in each polymerase chain reaction is the product obtained in the previous polymerase chain reaction, and all of the fragments of the target polynucleotide sequence used in the polymerase chain reactions in sequence constitute the target polynucleotide sequence. The target polynucleotide is synthesized in fragments during each polymerase chain reaction through un-annealed parts of the primer for extension by the primer extension technique. The advantage of the invention is that the product obtained in the previous polymerase chain reaction is directly taken as the template used in the afterward reaction without a purification step or other specific processing steps. The labor and time are less than conventional methods.

According to the invention, the primer pairs used in the polymerase chain reactions are constructed by extending the target polynucleotide at a direction from the 3′-end to the 5′-end (as shown in FIG. 1, right), and/or at a direction from the 5′-end to the 3′-end (as shown in FIG. 1, left). In one preferable embodiment of the invention, the target polynucleotide is extended at two directions, i.e. from the 3′-end to the 5′-end and from the 5′-end to the 3′-end of the target polynucleotide sequence (as shown in FIG. 2).

In one embodiment of the invention, the extension is conducted at the direction from the 3′-end to the 5′-end of the target polynucleotide sequence as shown in FIG. 1, right. The forward primer of the primer pair is designed to have the following parts:
(a) the part (a1), locating at the 5′-end region of the forward primer, comprising a fragment having more than 10 nucleotides and being homologous to the fragment at the 3′-end region of the target polynucleotide sequence, and
(b) the part (b1), locating at the 3′-end region of the forward primer, comprising a fragment having more than 10 nucleotides and being homologous to the sequence of the more than 10 nucleotides from the 5′-end region of the template sequence;
and wherein the 3′-end of the part (a1) is adjacent to the 5′-end of the part (b1).

[The part (a1) is designed for extending the target polynucleotide sequence, and the part (b1) is for annealing the template enabling DNA polymerase to catalyze DNA synthesis.]

The reversed primer is designed to have, at the 3′-end region of the reversed primer, a fragment having more than 5 nucleotides and being capable of annealing to the 3′-end region of the template sequence. Preferably, the reversed primer (6) used in the step (2) is the same as the reversed primer (2) of the first primer pair used in the step (1).

In another embodiment of the invention, the extension is conducted at the direction from the 5′-end to the 3′-end of the target sequence as shown in FIG. 1, left. The forward primer of the primer pair is designed to have at the 3′-end region, a fragment having more than 5 nucleotides and being homologous to the 5′-end region of the template sequence.

The reversed primer is designed to have the following parts:
(a) the part (a2), locating at the 5′-end region of the reversed primer, comprising a fragment having more than 10 nucleotides and being complement to the 5′-end region sequence of the target polynucleotide sequence;
(b) the part (b2), locating at the 3′-end region of the reversed primer, comprising a fragment having more than 10 nucleotides and be capable of annealing to the sequence of the more than 10 nucleotides from the 3′-end region of the template sequence,
and wherein the 3′-end of the part (a2) is adjacent to the 5′-end of the part (b2).
   [The part (a2) is designed for extending the target polynucleotide sequence, and the part (b2) is for annealing the template enabling DNA polymerase to catalyze DNA synthesis.]

The forward primer is designed for conducting the polymerase chain reactions. Preferably, the forward primer (7) used in the method (ii) of the step (2) is the same to the forward primer (3) of the first primer pair used in the step (1).

In the other embodiment of the invention, the extension is conducted at the both directions from the 3′-end to the 5′-end and from the 5′-end to the 3′-end of the target sequence as shown in FIG. 2. The forward primer of the primer pair is designed to have the following parts:
(a) the part (a3), locating at the 5′-end region, comprising a fragment having more than 10 nucleotides and being homologous to the fragment at the 3′-end region of the target polynucleotide sequence;
(b) the part (b3), locating at the 3′-end region of the forward primer, comprising a fragment having more than 10 nucleotides and being homologous to the sequence of the more than 10 nucleotides from the 5′-end region of the template sequence;
and wherein the 3′-end of the part (a3) is adjacent to the 5′-end of the part (b3).

The reversed primer is designed to have the following parts:
(c) the part (c3), locating at the 5′-end region, comprising a fragment having more than 10 nucleotides and being complement to the 5′-end region of the target polynucleotide sequence;
(d) the part (d3), locating at the 3′-end region of the reversed primer, comprising a fragment having more than 10 nucleotides and annealing to the sequence of the more than 10 nucleotides from the 3 ′-end region of the template sequence;
   and wherein the 3′-end of the part (c3) is adjacent to the 5′-end of the part (d3).

The parts (a3) and (c3) are designed for extending the target polynucleotide sequence, and the parts (b3) and (d3) are designed for annealing the template enabling DNA polymerase to catalyze DNA synthesis. If the first template is not relevant to the target polynucleotide, the 3′-end of the part (a3) and the 3′-end of the part (c3) are designed to be adjacent to each other in the target polynucleotide sequence. For the reason, the target polynucleotide sequence can be generated without disruption or discontinuation by the removal of the first template. If the first template is a fragment of the target polynucleotide, the 3′-end of the part (a3) is designed to be adjacent to the 5′-end of the first template in the target polynucleotide sequence, and the 3′-end of the part (c3) is designed to be adjacent to the 3′-end of the first template in the target polynucleotide sequence for constituting the target polynucleotide.

According to the invention, the primer sequences can be easily determined by artisans skilled in this field, and the conditions and the step of conducting the multi-cyclic polymerase chain reaction can be designed by artisans skilled in this field. Preferably, each primer used in each step is a fragment having more than 10 nucleotides, most preferably more than 15 nucleotides.

According to the invention, the method comprises the step (3) of obtaining the polynucleotide product comprising the target polynucleotide sequence from the final product of the muti-cyclic polymerase chain reactions.

According to the invention, the method further comprises a step of removing the nucleotide sequence of the first template from the final product in the step (3) so as to obtain the target polynucleotide sequence if the first template is irrelevant to the target polynucleotide sequence, such as the template sequence commonly used in the host-vector expression system. It can be achieved by an enzyme-digesting step or other conventional methods. Preferably, the first template is designed to have restriction enzyme recognition sites at the both ends, which are convenient to manipulation hereafter.

The present invention also provide a method for highly expressing a target heterogeneous polypeptide encoded by a target polynucleotide in a host, which comprises the steps of:
(1) providing a target polynucleotide obtained by the method for synthesizing a target polynucleotide that is efficiently expressed in a host-vector expression system as described above;
(2) transforming or transfecting the target polynucleotide to the host; and
(3) expressing the target heterogeneous protein in the transformed and transfected host.

According to the invention, the codons of the fragments of the target polynucleotide used for expressing the target heterogeneous polypeptide may be changed according to the data provided in the Wisconsin Package (The Wisconsin Package, by Genetics Computer Group, Inc. (1992)). For instance, the codon CTA encoding leucine may be changed to CTG, CTT, CTC, TTG, or TTA; the codon ATA encoding isoleucine may be changed to ATC or ATT; the codons CGG, AGG, AGA encoding arginine may be changed to CGT or CGC; the codon GGA encoding glycine changed to GGT or GGC; the codon CCC encoding proline may be changed to CCG, CCA or CCT; the codon CTA encoding leucine may be changed to CTG, CTT, CTC, TTG, or TTA; the codon ATA encoding isoleucine may be changed to ATC or ATT; the codons CGG, AGG, AGA encoding arginine may be changed to CGT or CGC; the codon GGA encoding glycine may be changed to GGT or GGC; or the codon CCC encoding proline may be changed to CCG, CCA or CCT, in order to enhance the translation rate.

In one embodiment of the invention, the method of transforming or transfecting the target polynucleotide to the host used in the step (2) may be any conventional method for introducing the polynucleotide into the host. Preferably, the polynucleotide having the target sequence are incorporated into a vector.

In one embodiment of the invention, the conditions for highly expressing the target heterogeneous protein in the transformed or transfected host used in the step (3) may be determined by artisans skilled in this field according to the properties of the heterogeneous protein and the host cell.

According to the invention, a heterogeneous protein can be translated by the target polynucleotide in a host; therefore, the problem of the low yield of expressing a heterogeneous protein in a host is solved.

The following Examples are given for the purpose of illustration only and are not intended to limit the scope of the present invention.

### EXAMPLE 1

### Method I for Synthesizing a Target Polynucleotide Encoding PRRSV-ORF7 Protein Efficiently Expressed in Escherichia coli

Target polynucleotide sequence: PRRSV-ORF 7 is a gene encoding a nucleocapsid protein in porcine reproductive and respiratory syndrome virus (PRRSV), and the sequence of the gene was obtained from National Center Biotechnology Information (http://www.ncbi.nlm.nih.gov). In the sequence, the codon CTA encoding leucine was changed to CTG, CTT, CTC, TTG, or TTA; the codon ATA encoding isoleucine to ATC or ATT, the codons CGG, AGG, AGA encoding arginine to CGT or CGC; the codon GGA encoding glycine to GGT pr GGC; and the codon CCC encoding proline to CCG, CCA or CCT according to the table of the codons for a high expression in E. coli in the Wisconsin Package. The changes of the codons were listed in Table 1.

A gene encoding PRRSV-ORF 7 in PRRSV was then designed as a target polynucleotide sequence as shown in SEQ ID NO: 1 for highly expressed in E. coli.

TABLE 1

| Aa | Condon | Number¹ | /1000² | Fraction³ |
|---|---|---|---|---|
| Gly | GGG | 13 | 1.89 | 0.02 |
| Gly | GGA | 3 | 0.44 | 0.00 |
| Gly | GGU | 365 | 52.99 | 0.59 |
| Gly | GGC | 238 | 34.55 | 0.38 |
| Glu | GAG | 108 | 15.68 | 0.22 |
| Glu | GAA | 394 | 57.20 | 0.78 |
| Asp | GAU | 149 | 21.63 | 0.33 |
| Asp | GAC | 298 | 43.26 | 0.67 |
| Val | GUG | 93 | 13.50 | 0.16 |
| Val | GUA | 146 | 21.20 | 0.26 |
| Val | GUU | 289 | 43.26 | 0.51 |
| Val | GUC | 38 | 5.52 | 0.07 |
| Ala | GCG | 161 | 23.37 | 0.26 |
| Ala | GCA | 173 | 25.12 | 0.28 |
| Ala | GCU | 212 | 30.78 | 0.35 |
| Ala | GCC | 62 | 9.00 | 0.10 |
| Arg | AGG | 1 | 0.15 | 0.00 |
| Arg | AGA | 0 | 0.00 | 0.00 |
| Ser | AGU | 9 | 1.31 | 0.03 |
| Ser | AGC | 71 | 10.31 | 0.20 |
| Lys | AAG | 111 | 16.11 | 0.26 |
| Lys | AAA | 320 | 46.46 | 0.74 |
| Asn | AAU | 19 | 2.76 | 0.06 |
| Asn | AAC | 274 | 39.78 | 0.94 |
| Met | AUG | 170 | 24.68 | 1.00 |
| Ile | AUA | 1 | 0.15 | 0.00 |
| Ile | AUU | 70 | 10.16 | 0.17 |
| Ile | AUC | 345 | 50.09 | 0.83 |
| Thr | ACG | 25 | 3.63 | 0.07 |
| Thr | ACA | 14 | 2.03 | 0.04 |
| Thr | ACU | 130 | 18.87 | 0.35 |
| Thr | ACC | 206 | 29.91 | 0.55 |
| Trp | UGG | 55 | 7.98 | 1.00 |
| Stop | UGA | 0 | 0.00 | (Stop) |
| Cys | UGU | 22 | 3.19 | 0.49 |
| Cys | UGC | 23 | 3.34 | 0.51 |
| Stop | UAG | 0 | 0.00 | (Stop) |
| Stop | UAA | 0 | 0.00 | (Stop) |
| Tyr | UAU | 51 | 7.4 | 0.25 |
| Tyr | UAC | 157 | 22.79 | 0.75 |
| Leu | UUG | 18 | 2.61 | 0.03 |
| Leu | UUA | 12 | 1.74 | 0.02 |
| Phe | UUU | 51 | 7.4 | 0.24 |
| Phe | UUC | 166 | 24.10 | 0.76 |
| Ser | UCG | 14 | 2.03 | 0.04 |
| Ser | UCA | 7 | 1.02 | 0.02 |
| Ser | UCU | 120 | 17.42 | 0.34 |
| Ser | UCC | 131 | 19.02 | 0.37 |
| Arg | CGG | 1 | 0.15 | 0.00 |
| Arg | CGA | 2 | 0.29 | 0.01 |
| Arg | CGU | 290 | 42.10 | 0.74 |
| Arg | CGC | 96 | 13.94 | 0.25 |
| Gln | CAG | 233 | 33.83 | 0.86 |
| Gln | CAA | 37 | 5.37 | 0.14 |
| His | CAU | 18 | 2.61 | 0.17 |
| His | CAC | 85 | 12.34 | 0.83 |
| Leu | CUG | 480 | 69.69 | 0.83 |
| Leu | CUA | 2 | 0.29 | 0.00 |
| Leu | CUU | 25 | 3.63 | 0.04 |
| Leu | CUC | 38 | 5.52 | 0.07 |
| Pro | CCG | 190 | 27.58 | 0.77 |
| Pro | CCA | 36 | 5.23 | 0.15 |
| Pro | CCU | 19 | 2.76 | 0.08 |
| Pro | CCC | 1 | 0.15 | 0.00 |

¹Number of occurrences of the codon in the genes from which the table is compiled.
²Expected number of occurrences per 1000 codon in genes whose codon usage is identical to that compiled in the frequency table.
³Fraction of occurrences of the codon in synonymous codon family.

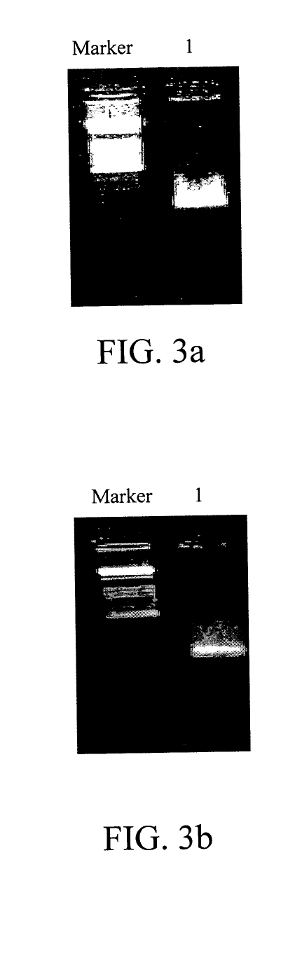

First Template. The wild type PRRSV genome was taken for generating the first template. In order to clone the target polynucleotide sequence (SEQ ID NO: 1) in an expression plasmid, pET23a, a forward primer, ORF7-pET23a-Nde I-F, 5′-CCGCGCGGCAGCCATATGCCAAATAACAAC-3′ (SEQ ID NO: 2) comprising a cloning site in its 5′-portion and first 18 nucleotides of SEQ ID NO: 1 in its 3′-portion along with a reversed primer, ORF7-C-R0, 5′-CTTCTTATTTTTACTACCCGGTCCCTTAACTCTGGA-3′ (SEQ ID NO: 3) for changing the codons GGA and AGG to GGT and AGT were used. The polymerase chain reaction was carried out with adding Pfu polymerase, dNTP and reaction buffer. The thermocycle was 5 minutes at 95° C. followed by 20 cycles of 1 minute at 94° C., 30 seconds at 55° C. and 1 minute at 72° C. The product was subjected to agarose gel electrophoresis. The result was shown in FIG. 3a, lane 1. The product was further taken together with the forward primer, ORF7-pET23a Nde I-F (SEQ ID NO: 2) and a reversed primer ORF7-C-R1, 5′-CTTCTTATTTTTACGACCCGGACCCTTAACACGGGA-3′ (SEQ ID NO: 4) to carry out another polymerase chain reaction as described above. In the ORF7-C-R1, the codons AGA and GGA to CGT and GGT were changed. The product obtained was used as the first template and the result of agarose gel electrophoresis was shown in FIG. 3b, lane 1.

First Primer Pair: The extension was in a direction of from the 5′-end to the 3′-end of the target sequence. The forward primer ORF7-pET23a-Nde I-F (SEQ ID NO: 2) and a reversed primer ORF7-C-R2, 5′-TGCGGCTTCTCCGGGTTTTTCTTCTTATTTTTACG-3′ (SEQ ID NO: 5) were as the first primer pair. The 3′-portion of ORF7-C-R2 was used for annealing 129 to 141 nt of SEQ ID NO: 1, and the 5′-portion was for generating the 142 to 164 nt of SEQ ID NO: 1.

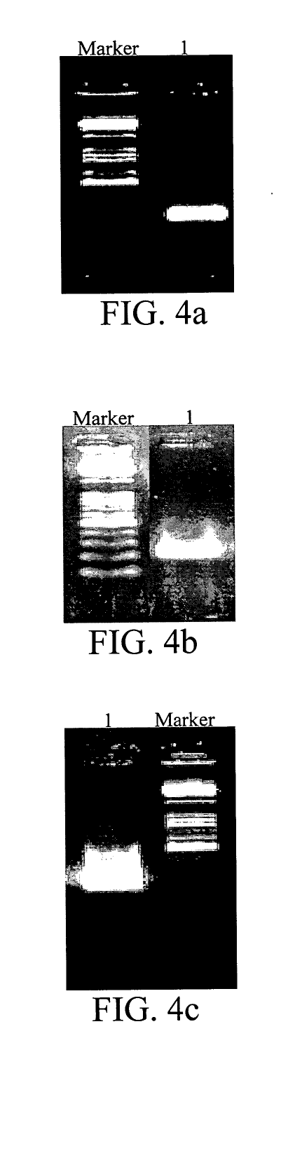

First Polymerase Chain Reaction Product: One μL of the first template, 4 μL of each of the first primer pair, dNTP, reaction buffer, and Pfu polymerase were mixed to conduct a multi-cyclic polymerase chain reaction. The thermocycle was 5 minutes at 95° C. followed by 20 cycles of 1 minute at 94° C., 30 seconds at 55° C. and 1 minute at 72° C. The product was subjected to agarose gel electrophoresis. The result was shown in FIG. 4a, lane 1.

Second Primer Pair: The forward primer ORF7-pET23a-Nde I-F (SEQ ID NO: 2) and a reversed primer ORF7-C-R3, 5′-GTCGCCAGAGGAAAATGCGGCTTCTCCGGGTTT-3′ (SEQ ID NO: 6) were used as a second primer pair. The (b2) part (3′-end region) of ORF7-C-R3 was used for annealing to the first polymerase chain reaction product as shown in 147 to 164 nt of SEQ ID NO: 1, and the (a2) part (5′-end region) was for generating the 165 to 179 nt of SEQ ID NO: 1.

Polymerase Chain Reaction Product. One μL of the first polymerase chain reaction product, 4 μL of each of the second primer pair, dNTP, reaction buffer, and Pfu polymerase were mixed to conduct a multi-cyclic polymerase chain reaction. The thermocycle was 5 minutes at 95° C. followed by 20 cycles of 1 minute at 94° C., 30 seconds at 55° C. and 1 minute at 72° C. The product was subjected to agarose gel electrophoresis. The result was shown in FIG. 4b, lane 1.

Third Primer Pair: The forward primer ORF7-pET23a-Nde I-F (SEQ ID NO: 2) and a reversed primer ORF7-C-R4, 5′-TGGTGACGGACGTCATCTTCAGTCGCCAGAGG-3′ (SEQ ID NO: 7) were as the third primer pair. The (b2) part (3′-end region) of ORF7-C-R4 was used for annealing the second polymerase chain reaction product as shown in 169 to 179 nt of SEQ ID NO: 1, and the (a2) part (5′-end region) was used for generating the 180 to 200 nt of SEQ ID NO: 1.

Polymerase Chain Reaction Product: One μL of the polymerase chain reaction product of the third second primer pair, μL of each of the new second primer pair, dNTP, reaction buffer, and Pfu polymerase were mixed to conduct a multi-cyclic polymerase chain reaction. The thermocycle was 5 minutes at 95° C. followed by 20 cycles of 1 minute at 94° C., 30 seconds at 55° C. and 1 minute at 72° C. The product was subjected to sequence analysis and agarose gel electrophoresis. The electrophoresis result was shown in FIG. 4c, lane 1. Therefore, the target polynucleotide whose sequence as shown in SEQ ID NO: 1 was obtained.

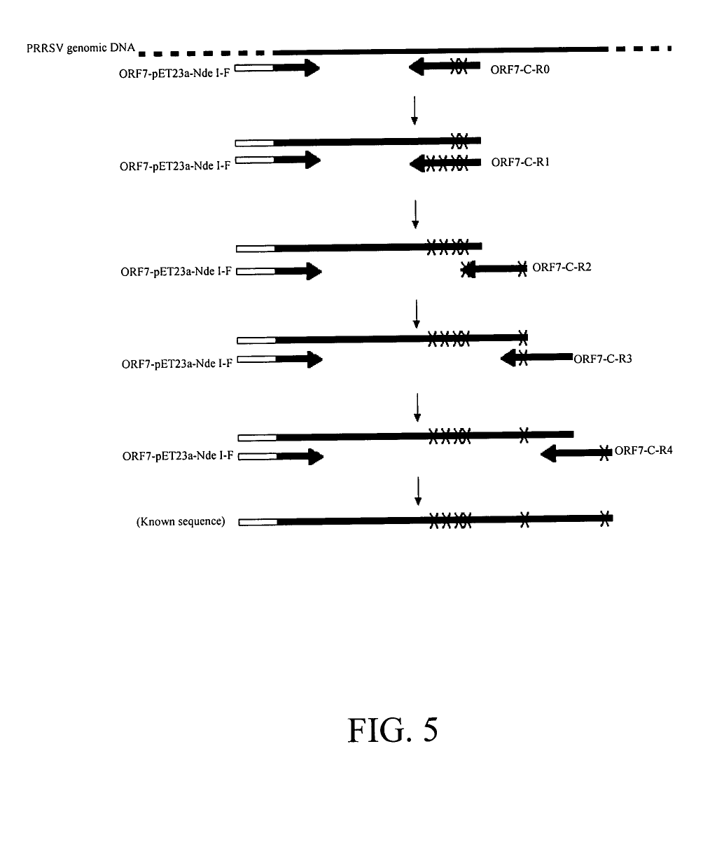

The schematic figure of the example was shown in FIG. 5.

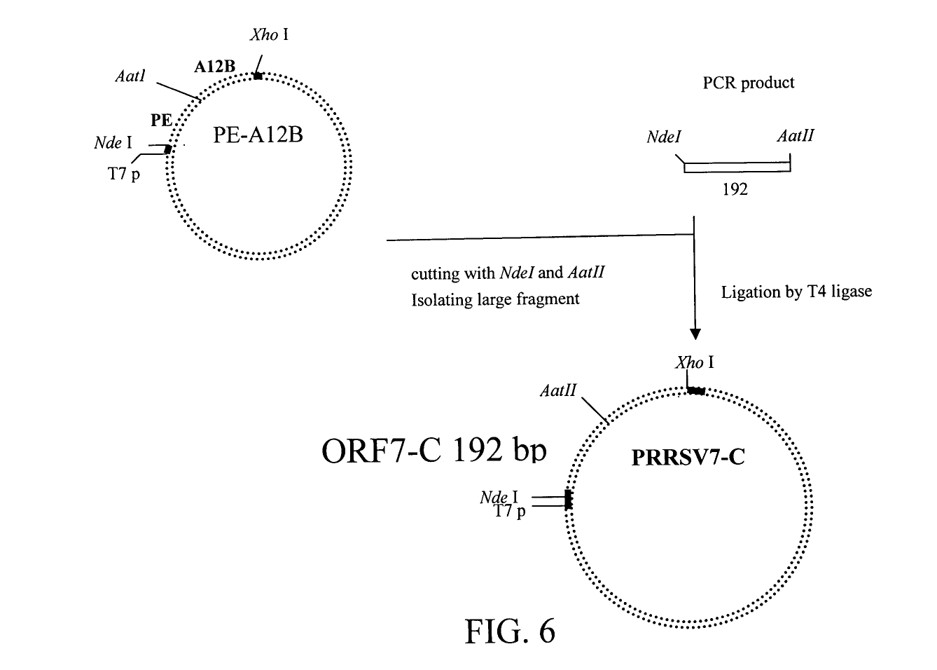

Cloning: The polymerase chain reaction product and PE-A12B plasmid with a backbone of pET23a were both digested with restriction enzymes Nde I and Aat II. The larger fragments were taken and ligated together to obtain the plasmid PPRSV7-C by T4 ligase (as shown in FIG. 6).

Expressing: The PRRSV7-C plasmid was transformed to JM109 competent cells for expression.

### EXAMPLE 2

### Method II for Synthesizing a Target Polynucleotide Encoding PRRSV-ORF7 Protein Efficiently Expressed in Escherichia coli

Example 2 provides a method for synthesizing PRRSV-ORF7, which is similar to Example 1 with some modifications.

The target polynucleotide sequence (SEQ ID NO: 1) in the example was as described in Example 1, and the first template was a part of the wild type PRRSV genome.

First Primer Pair: The first primer pair used in the example was ORF7-pET23a-Nde I-F (SEQ ID NO: 2) and ORF7-C-R0 (SEQ ID NO: 3). A helper primer ORF7-C-R1 (SEQ ID NO: 4) was also provided. ORF7-C-R0 and ORF7-C-R1 were in the ratio of 1:19. The 3′-end region of ORF7-C-R0 was used for annealing the first template, and the 5′-end region was for generating a part of the target polynucleotide sequence shown in SEQ ID NO: 1. The 3′-end region of ORF7-C-R1 was also part of SEQ ID NO: 1 and can anneal the product made by the ORF7-pET23a-Nde I-F and ORF7-C-R0.

First Polymerase Chain Reaction Product: One μL of the adapter template polymerase, 4 μL of ORF7-pET23a-Nde I-F, 0.2 μL of ORF7-C-R0, 3.8 μL of ORF7-C-R1, dNTP, reaction buffer, and Pfu polymerase were mixed to conduct a multi-cyclic polymerase chain reaction. The thermocycle was 5 minutes at 95° C. followed by 20 cycles of 1 minute at 94° C., 30 seconds at 55° C. and 1 minute at 72° C. The product was subjected to agarose gel electrophoresis. The product was as the first template in Example 1 and the steps hereafter of synthesizing PRRSV-ORF7 were similar to the description in Example 1.

### EXAMPLE 3

### Method of Synthesizing a Target Polynucleotide Encoding FMD-vpg Protein Efficiently Expressed in E. coli

Target Polynucleotide Sequence: FMD-vpg (3828-5975) was a gene encoding a non-structural protein of Taiwanese foot-and-mouth disease (FMD) virus, and the sequence of the gene was reported by Beard et al.(Beard, C. W. and Mason, P. W. 2000. Genetic determinants of altered virulence of Taiwanese foot-and-mouth disease virus J. Virol 74 (2), 987-991). In the FMD-vpg, the codon GGA encoding glycine was changed to GGT; the codon AGA encoding leucine to CGT; and the codon ATA encoding isoleucine to ATC to enhance the expression of the protein in an enteric bacterium (see Table 1). A gene encoding FMD-vpg in Taiwanese foot-and-mouth disease virus was then designed to a target polynucleotide sequence as shown in SEQ ID NO: 8 for highly expressed in E. coli.

First Template: A part of pET-23a (SEQ ID NO: 9) which was a template sequence commonly used in the host-vector expression system was taken as the first template, where the sequence was known and had cloning sites for manipulation.

First Primer Pair. The extension was toward the 5′-end of the SEQ ID NO: 10. 3B-F1, 5′-TTGATCGTCACTGAGGTCGACAAGCTTGCG-3′ (SEQ ID NO: 10) and a reversed primer T7t-R1, 5′-TTATGCTAGTTATTGCTCAGCGGTGGCAGC-3′ (SEQ ID NO: 11) were as the first primer pair. The 3′-end region of 3B-F1 was used for annealing the 1 to 15 nt of SEQ ID NO: 9, and the 5′-end region was for generating the 148 to 162 nt of SEQ ID NO: 8.

First Polymerase Chain Reaction Product: One μL of the first template, 4 μL of each of the first primer pair, dNTP, reaction buffer, and Pfu polymerase were mixed to conduct a multi-cyclic polymerase chain reaction. The thermocycle was 5 minutes at 95° C. followed by 20 cycles of 1 minute at 94° C., 30 seconds at 55° C. and 1 minute at 72° C.

Second Primer Pair: T7t-R1 (SEQ ID NO: 11) and a forward primer 3B-F2, 5′-AGTGAAAGCAAAGAACTTGATCGTCACTGAG-3′ (SEQ ID NO: 12) were as the second primer pair. The (b1) part (3′-end region) of 3B-F2 was used for annealing the first polymerase chain reaction product as 148 to 162 nt of SEQ ID NO: 8, and the (a1) part (5′-end region) was for generating the 132 to 147 nt of SEQ ID NO: 8.

Polymerase Chain Reaction Product: One μL of the second polymerase chain reaction product, 4 μL of each of the second primer pair, dNTP, reaction buffer, and Pfu polymerase were mixed to conduct a multi-cyclic polymerase chain reaction. The thermocycle was 5 minutes at 95° C. followed by 20 cycles of 1 minute at 94° C., 30 seconds at 55° C. and 1 minute at 72° C.

Primer Pair. T7t-R1 (SEQ ID NO: 11) and a serious of forward primers were used as the primer pair sequentially:

(SEQ ID NO: 13)

| | |
|---|---|
| 3B-F3: | 5′-ACCTGTCGCTTTGAAAGTGAAAGCAAAGAAC-3′; |

(SEQ ID NO: 14)

| | |
|---|---|
| 3B-F4: | 5′-GGTCCGGTGAAGAAACCTGTCGCTTTGAAA-3′; |

(SEQ ID NO: 15)

| | |
|---|---|
| 3B-F5: | 5′-GAAGGTCCTTACGAGGGTCCGGTGAAGAAA-3′; |

(SEQ ID NO: 16)

| | |
|---|---|
| 3B-F6: | 5′-AAAGCCCCGGTCGTGAAGGAAGGTCCTTACGAG-3′; |

(SEQ ID NO: 17)

| | |
|---|---|
| 3B-F7; | 5′-ACCGCTGAAGGTGAAAGCAAAAGCCCCGGTCGTG-3′; |

(SEQ ID NO: 18)

| | |
|---|---|
| 3B-F8: | 5′-CCAATGGAGCGTCAGAAACCGCTGAAGGTGAAA-3′; |

(SEQ ID NO: 19)

| | |
|---|---|
| 3B-F9; | 5′-GAGGGTCCATACGCCGGCCCAATGGAGCGTCAGA-3′; |

(SEQ ID NO: 20)

| | |
|---|---|
| 3B-F10; | 5′-AAAAAATCCCATATGGAGGGTCCATACGCC-3′. |

The (b1) parts (3′-end regions) of the 3B-F3 to 3B-F10 were used for annealing the former polymerase chain reaction product, and the (a1) parts (5′-end regions) were used for generating the polynucleotide whose sequence was shown in SEQ ID NO: 8. The condition of conducting the polymerase chain reaction was similar to the condition used for conducting the first chain reaction product.

Cloning: The polymerase chain reaction product and an IPTG inductive expression plasmid were both digested with restriction enzymes Xho I and Nde I. The larger fragments were taken and ligated together by T4 ligase.

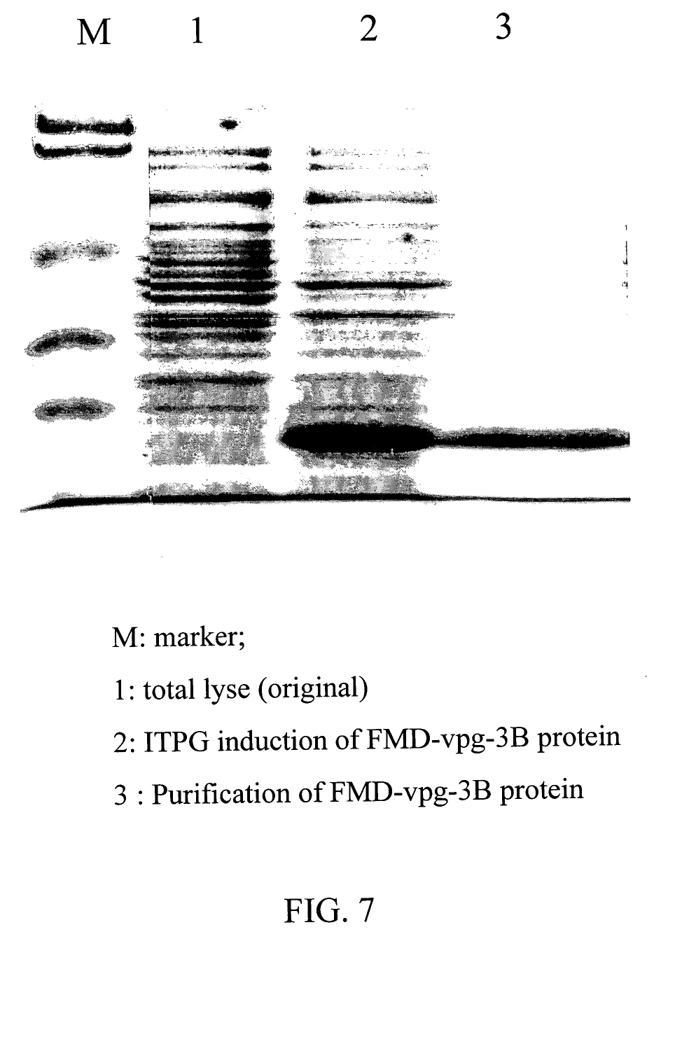

Expression: The vector obtained was transformed to JM109 competent cells for expression. FMD-vpg-3B protein was expressed and purified for resolving in SDS-PAGE and shown in FIG. 7. It showed that the protein with some codon changes could be expressed in a high level with the induction of IPTG.

### EXAMPLE 4

### Primer Length for Carrying Out Polymerase Chain Reaction

Primers with different lengths were taken for carrying out polymerase chain reaction. The primer design and method for polymerase chain reaction were similar to those in Example 1. The primer pair was listed in Table 2.

TABLE 2

| | | Length of 3′-end | Length of 5′-end |
|---|---|---|---|
| Primer pair | Length of primer | region | region |
| 1 | 16 | 8 | 8 |
| 2 | 20 | 10 | 10 |
| 3 | 24 | 12 | 12 |
| 4 | 30 | 15 | 15 |
| 5 | 25 | 10 | 15 |

The result of polymerase chain reaction was shown in Table 3, and wherein “+” refers to that a desired fragment was observed in an agarose gel electrophoresis; “−” refers to that a desired fragment was not observed in an agarose gel electrophoresis. It showed that the lengths of the 3′-end region and the 5′-end region should be both more than 10 nucleotides.

TABLE 3

Primer pair

| | 1 | 2 | 3 | 4 | 5 |
|---|---|---|---|---|---|
| Result | − | − | + | + | + |

While embodiments of the present invention have been illustrated and described, various modifications and improvements can be made by persons skilled in the art. The embodiments of the present invention are therefore described in an illustrative but not restrictive sense. It is intended that the present invention is not limited to the particular forms as illustrated, and that all the modifications not departing from the spirit and scope of the present invention are within the scope as defined in the appended claims.

1. A method for synthesizing a target polynucleotide that is efficiently expressed in a host-vector expression system, comprising the steps of
(1) conducting a first polymerase chain reaction on a first template with a first primer pair to obtain a first polymerase chain reaction product; which is characterized in that the first template is any template sequence commonly used in the host-vector expression system or a fragment of the target polynucleotide;
(2) conducting muti-cyclic polymerase chain reactions by a primer extension technique to obtain a product comprising the target polynucleotide sequence; wherein the template used in each polymerase chain reaction is the product obtained in the previous polymerase chain reaction; and
which is characterized in that the primer pairs used in the polymerase chain reactions are designed to be any one of the following three primer pairs:
(i) the forward primer having two parts:
(a) the part (a1), locating at the 5 end region of the forward primer, comprising a fragment having more than 10 nucleotides and being homologous to the fragment at the 3 end region of the target polynucleotide sequence, and
(b) the part (b1), locating at the 3 end region of the forward primer, comprising a fragment having more than 10 nucleotides and being homologous to the sequence of the more than 10 nucleotides from the 5 end region of the template sequence;
and wherein the 3 end of the part (a1) is adjacent to the 5 end of the part (b1); and
the reversed primer having, at the 3 end region of the reversed primer, a fragment having more than 5 nucleotides and being capable of annealing to the 3 end region of the template sequence;
(ii) the forward primer having at the 3 end region of the forward primer, a fragment having more than 5 nucleotides and being homologous to the 5 end region of the template sequence; and
the reversed primer having
(a) the part (a2), locating at the 5 end region of the reversed primer, comprising a fragment having more than 10 nucleotides and being complement to the 5 end region sequence of the target polynucleotide sequence;
(b) the part (b2), locating at the 3 end region of the reversed primer, comprising a fragment having more than 10 nucleotides and be capable of annealing to the sequence of the more than 10 nucleotides from the 3 end region of the template sequence,
and wherein the 3 end of the part (a2) is adjacent to the 5 end of the part (b2); and
(iii) the forward primer having
(a) the part (a3), locating at the 5 end region of the forward primer, comprising a fragment having more than 10 nucleotides and being homologous to the fragment at the 3 end region of the target polynucleotide sequence;
(b) the part (b3), locating at the 3 end region of the forward primer, comprising a fragment having more than 10 nucleotides and being homologous to the sequence of the more than 10 nucleotides from the 5 end region of the template sequence and wherein the 3 end of the part (a3) is adjacent to the 5 end of the part (b3); and the reversed primer having
(c) the part (c3), locating at the 5 end region of the reversed primer, comprising a fragment having more than 10 nucleotides and being complement to the 5 end region of the target polynucleotide sequence;
(d) the part (d3), locating at the 3 end region of the reversed primer, comprising a fragment having more than 10 nucleotides and annealing to the sequence of the more than 10 nucleotides from the 3 end region of the template sequence; and wherein the 3 end of the part (c3) is adjacent to the 5 end of the part (d3); and wherein all of the fragments of the target polynucleotide sequence used in the polymerase chain reactions in sequence constitute the target polynucleotide sequence; and
(3) obtaining the polynucleotide product comprising the target polynucleotide sequence from the final product of the muti-cyclic polymerase chain reactions.

2. The method according to claim 1, further comprising a step of removing the nucleotide sequence of the first template from the final product in the step (3) so as to obtain the target polynucleotide sequence if the first template is irrelevant to the target polynucleotide sequence.

3. The method according to claim 2, wherein the first template is designed to have restriction enzyme recognition sites at the both ends.

4. The method according to claim 1, wherein the fragment having more than 10 nucleotides used in each step is more than 15 nucleotides.

5. The method according to claim 1, wherein if the target polynucleotide sequence is heterogeneous to the host used in expressing the protein encoding the target polynucleotide, some codons of the target polynucleotide are changed to the codons which have a high expression efficiency in translating the same amino acid in the host cell.

6. The method according to claim 1, wherein the host is an enteric bacterium.

7. A method for highly expressing a target heterogeneous polypeptide encoded by a target polynucleotide in a host, which comprises the steps of:
(1) providing a target polynucleotide obtained by the method according to claim 1;
(2) transforming or transfecting the target polynucleotide to the host; and
(3) expressing the target heterogeneous protein in the transformed or transfected host.

8. The method according to claim 7, wherein the host is an enteric bacterium.

9. The method according to claim 8, which further comprises, in the fragments of the target polynucleotide used for expressing the target heterogeneous polypeptide, changing the codon CTA encoding leucine to CTG, CTT, CTC, TTG, or TTA; the codon ATA encoding isoleucine to ATC or ATT; the codons CGG, AGG, AGA encoding arginine to CGT or CGC; the codon GGA encoding glycine changed to GGT or GGC; the codon CCC encoding proline to CCG, CCA or CCT; the codon CTA encoding leucine to CTG, CTT, CTC, TTG, or TTA; the codon ATA encoding isoleucine to ATC or ATT; the codons CGG, AGG, AGA encoding arginine to CGT or CGC; the codon GGA encoding glycine to GGT or GGC; or the codon CCC encoding proline to CCG, CCA or CCT.

10. The method according to claim 1, wherein the target polynucleotide encodes a mutated protein which has multiple mutation sites comparing to the wild-type form thereof

11. The method according to claim 1, wherein the first polymerase chain reaction in the step (1) further conducted by a helper primer which is homologous to one primer of the first primer pair and identical to a fragment of one strand of the target polynucleotide.
Please add the following new claims:

12. A method for highly expressing a target heterogeneous polypeptide encoded by a target polynucleotide in a host, which comprises the steps of
(1) providing a target polynucleotide obtained by the method according to claim 2;
(2) transforming or transfecting the target polynucleotide to the host; and
(3) expressing the target heterogeneous protein in the transformed or transfected host.

13. A method for highly expressing a target heterogeneous polypeptide encoded by a target polynucleotide in a host, which comprises the steps of
(1) providing a target polynucleotide obtained by the method according to claim 3;
(2) transforming or transfecting the target polynucleotide to the host; and
(3) expressing the target heterogeneous protein in the transformed or transfected host.

14. A method for highly expressing a target heterogeneous polypeptide encoded by a target polynucleotide in a host, which comprises the steps of
(1) providing a target polynucleotide obtained by the method according to claim 4;
(2) transforming or transfecting the target polynucleotide to the host; and
(3) expressing the target heterogeneous protein in the transformed or transfected host.

15. A method for highly expressing a target heterogeneous polypeptide encoded by a target polynucleotide in a host, which comprises the steps of
(1) providing a target polynucleotide obtained by the method according to claim 5;
(2) transforming or transfecting the target polynucleotide to the host; and
(3) expressing the target heterogeneous protein in the transformed or transfected host.


SEQUENCE LISTING (20 sequences)

SEQ ID NO: 1 - DNA, 200 bp, Artificial
  a target polynucleotide for highly expressing PRRSV-ORF 7 in Escherichia coli
catatgccaaataacaacggcaagcagcagaagaaaaagaagggggacggccagccagtcaatcagctgtgccaaatgctgggtaagatcatcgcccagcaaagtcagtcccgtgttaagggtccggggcgtaaaaataagaagaaaaacccggagaagccgcattttcctctggcgactgaagatgacgtccgtcacca

SEQ ID NO: 2 - DNA, 30 bp, Artificial
  a forward primer
ccgcgcggcagccatatgccaaataacaac

SEQ ID NO: 3 - DNA, 36 bp, Artificial
  a reversed primer
cttcttatttttactacccggtcccttaactctgga

SEQ ID NO: 4 - DNA, 36 bp, Artificial
  a reversed primer
cttcttatttttacgacccggacccttaacacggga

SEQ ID NO: 5 - DNA, 35 bp, Artificial
  a reversed primer
tgcggcttctccgggtttttcttcttatttttacg

SEQ ID NO: 6 - DNA, 33 bp, Artificial
  a reversed primer
gtcgccagaggaaaatgcggcttctccgggttt

SEQ ID NO: 7 - DNA, 32 bp, Artificial
  a reversed primer
tggtgacggacgtcatcttcagtcgccagagg

SEQ ID NO: 8 - DNA, 162 bp, Artificial
  a target polynucleotide for highly expressing FMD-vpg in Escherichia coli
aaaaaatcccatatggagggtccatacgccggcccaatggagcgtcagaaaccgctgaaggtgaaagcaaaagccccggtcgtgaaggaaggtccttacgagggtccggtgaagaaacctgtcgctttgaaagtgaaagcaaagaacttgatcgtcactgag

SEQ ID NO: 9 - DNA, 123 bp, Artificial
  partial pET-23a plasmid
gtcgacaagcttgcggccgcactcgagcaccaccaccaccaccactgagatccggctgctaacaaagcccgaaaggaagctgagttggctgctgccaccgctgagcaataactagcataaccc

SEQ ID NO: 10 - DNA, 30 bp, Artificial
  a forward primer
ttgatcgtcactgaggtcgacaagcttgcg

SEQ ID NO: 11 - DNA, 30 bp, Artificial
  a reversed primer
ttatgctagttattgctcagcggtggcagc

SEQ ID NO: 12 - DNA, 31 bp, Artificial
  a forward primer
agtgaaagcaaagaacttgatcgtcactgag

SEQ ID NO: 13 - DNA, 31 bp, Artificial
  a forward primer
acctgtcgctttgaaagtgaaagcaaagaac

SEQ ID NO: 14 - DNA, 30 bp, Artificial
  a forward primer
ggtccggtgaagaaacctgtcgctttgaaa

SEQ ID NO: 15 - DNA, 30 bp, Artificial
  a forward primer
gaaggtccttacgagggtccggtgaagaaa

SEQ ID NO: 16 - DNA, 33 bp, Artificial
  a forward primer
aaagccccggtcgtgaaggaaggtccttacgag

SEQ ID NO: 17 - DNA, 34 bp, Artificial
  a forward primer
accgctgaaggtgaaagcaaaagccccggtcgtg

SEQ ID NO: 18 - DNA, 33 bp, Artificial
  a forward primer
ccaatggagcgtcagaaaccgctgaaggtgaaa

SEQ ID NO: 19 - DNA, 34 bp, Artificial
  a forward primer
gagggtccatacgccggcccaatggagcgtcaga

SEQ ID NO: 20 - DNA, 30 bp, Artificial
  a forward primer
aaaaaatcccatatggagggtccatacgcc